(19) United States
(12) Patent Application Publication
(10) Pub. No.: US 2010/0113758 A1
(43) Pub. Date: May 6, 2010

(54) METHOD FOR PURIFYING BIOMOLECULES

(75) Inventors: Friederike Wilmer, Hilden (DE); Anja Schultz, Hilden (DE); Claudia Dienemann, Hilden (DE); Andreas Schäfer, Hilden (DE)

Correspondence Address: Baker Donelson Bearman, Caldwell & Berkowitz, PC, 555 Eleventh Street, NW, Sixth Floor, Washington, DC, 20004, US

(73) Assignee: QIAGEN GMBH, 40724 Hilden (DE)

(21) Appl. No.: 12/594,589

(22) PCT Filed: Mar. 20, 2008
(86) PCT No.: PCT/EP2008/053375

§ 371 Date: Dec. 21, 2009

(30) Foreign Application Priority Data
Apr. 4, 2007 (DE) 10 2007 016 707.7

(51) Int. Cl. C07H 21/04; C07H 21/00; C12N 9/00; C12M 1/00; C07H 21/02; C12N 1/08

(52) U.S. Cl. 536/23.1; 536/25.41; 435/183; 435/283.1; 435/270

(57) ABSTRACT

The present invention relates to a process for the purification of biomolecules, in particular of nucleic acids, such as DNA and RNA molecules.

### FIELD OF THE INVENTION

The present invention relates to a process for the purification of biomolecules, in particular of nucleic acids, such as DNA and RNA molecules.

### TECHNICAL BACKGROUND

The purification and analysis of biomolecules from biological samples plays an ever greater role in fundamental biomedical research, clinical research and diagnostics, forensic analysis, research into population genetics, epidemiological analysis and specialist fields related to these. This applies in particular to nucleic acids, such as DNA and RNA molecules, but also to amino acids, oligopeptides, polypeptides, monosaccharides, oligosaccharides, polysaccharides, fats, fatty acids and/or lipids.

Biology has developed a comprehensive set of molecular biology instruments for this in the last twenty years. A still more widespread use of molecular biology analyses is therefore to be expected for the future, e.g. in medical and clinical diagnostics, in forensics, in pharmacy in the development and evaluation of medicaments, in foodstuff analysis and in the monitoring of foodstuff production, in agricultural science in the breeding of crop plants and stock animals and in environmental analysis and in many fields of research.

By analysis of the transcriptome, that is to say the mRNA in cells, the activities of genes can be determined directly. Quantitative analysis of transcript patterns (mRNA patterns) in cells by modern molecular biology methods, such as e.g. real time reverse transcriptase PCR (“real time RT PCR”) or gene expression chip analyses makes it possible e.g. to detect defectively expressed genes, as a result of which e.g. metabolic diseases, infections or any predisposition towards cancer disease can be detected.

Analysis of the genome, that is to say the entire cell DNA, by molecular biology methods, such as e.g. PCR, NASBA, RFLP, AFLP or sequencing, makes it possible e.g. to detect genetic defects or determine the HLA type and other genetic markers. DNA fingerprinting for forensic, population genetics or foodstuff legislation analysis moreover fall under this generic term. Analysis of genomic DNA and RNA is also employed for direct detection of infectious pathogens, such as viruses, bacteria etc.

Analysis of other biomolecules, such as e.g. amino acids, oligopeptides, polypeptides, monosaccharides, oligosaccharides, polysaccharides, fats, fatty acids and/or lipids, can provide e.g. information on particular physiological states, on contamination in foodstuffs, on the content of particular nutrients and so on.

A prerequisite of all these approaches is, however, that the biomolecules, in particular nucleic acids, contained in a sample are isolated or purified so that they can subsequently be subjected to one of the subsidiary processes described.

Since the biomolecules to be detected often occur only in a very low concentration, effective and high-yield purification of the biomolecules contained in the sample is of decisive importance.

There is a large number of processes for purification of biomolecules from biological samples. A centrifugation step is often used here, in the context of which a dissolved sample is introduced into a centrifuge vessel containing a binding matrix. During centrifugation the solution is conveyed through the matrix and the biomolecules to be purified remain on the matrix in a bound form. During the subsequent course of the procedure, they are then eluted from the matrix and collected.

A process for the purification of nucleic acids which follows this principle is e.g. the so-called “boom principle” process disclosed in EP389063.

In this, a sample containing nucleic acids is introduced into a vessel with a silicate matrix in the presence of a chaotropic salt. The vessel is then centrifuged, or a vacuum is applied. This causes the nucleic acids to bind to the silicate matrix, while all the other constituents of the sample (in particular cell debris, organelles, proteins and the like) pass through the silicate matrix and are discarded. The bound nucleic acids are then eluted with a suitable agent and subjected to further analysis.

The mechanisms relevant to the binding are described e.g. in Melzak et al. (1996), Driving Forces for DNA Adsorption to Silica in Perchlorate Solutions, Journal of Colloid and Interface Science 181 (2), 635-644.

So-called “spin columns” are often used for this process. These are microreaction vessels which contain a disk-like silicate matrix, are open at the bottom, and are positioned in a further microreaction vessel closed at the bottom. The sample containing nucleic acids is pipetted into the microreaction vessel together with a chaotropic salt. The combination of the two microreaction vessels is then introduced into a centrifuge and centrifuged at an acceleration value of about 10000×g. During this procedure the nucleic acids bind to the silicate matrix, while all the other constituents of the sample pass through the silicate matrix and are transferred into the second microreaction vessel closed at the bottom. The latter is then discarded, while the bound nucleic acids are eluted with a suitable agent and subjected to further analysis.

Such and similar products are available inter alia from the applicant of the present invention, but also from competitors, such as Promega, Ambion, Macherey and Nagel and Invitrogen.

A critical feature of such processes for purification of biomolecules is that the yields achieved are inadequate in many cases. These are cases in particular in which the amount of biomolecules in the sample is so low that the yield with conventional purification methods is not sufficient for the molecules subsequently to be detected. Such samples are e.g. forensic samples or samples in which the RNA of a weakly expressed gene is to be analyzed.

### OBJECT OF THE PRESENT INVENTION

The present invention is based on the object of overcoming the disadvantages described resulting from the prior art. In particular, it is an object of the present invention to improve the processes mentioned such that the yields of biomolecules achieved are increased so that biomolecules, in particular nucleic acids, can also be purified from a sample under adverse circumstances and can be made accessible for subsequent analysis.

### SUMMARY OF THE INVENTION

This object is achieved with the features of the main claim submitted. The sub-claims describe preferred embodiments. It is to be noted here that the given ranges stated are always to be understood as including the particular limit values.

It is accordingly envisaged to provide a process for the purification of biomolecules from a sample which comprises the following steps:
a) arrangement of a reaction vessel with a binding matrix in a centrifuge, wherein a solution or suspension of a sample containing biomolecules is prepared in the reaction vessel or introduced into the reaction vessel before or after this step; and
b) inclusion of at least one multi-stage centrifugation step comprising at least a first centrifugation step at a first acceleration value and at least a second centrifugation step at a second acceleration value which is higher than the first acceleration value; wherein
c) step b) can be a binding step, a washing step and/or an elution step.

Preferably, the multi-stage step b) is a binding step in which the biomolecules are bound to the binding matrix by centrifugation. Considerably improved yields of biomolecules are established in this case, as demonstrated in the examples. However, this step can likewise preferably also be a washing step.

It can furthermore be envisaged that optionally further centrifugation steps are included before the first, between the first and the second or after the second centrifugation step.

In a further preferred embodiment, it is moreover envisaged that the process comprises at least a binding step, a washing step and an elution step, which always comprise at least an optionally multi-stage centrifugation step.

Particularly preferably, it is envisaged that the biomolecules are substances chosen from the group containing nucleic acids, amino acids, oligopeptides, polypeptides, monosaccharides, oligosaccharides, polysaccharides, fats, fatty acids and/or lipids.

In the following, the term “nucleic acids” is to be understood as meaning in particular RNA and DNA. Plasmid, genomic, viral and mitochondrial DNA in particular are possible here as DNA, while mRNA, siRNA, miRNA, rRNA, snRNA, t-RNA, hnRNA and total RNA in particular are possible as RNA.

In principle, the nucleic acids introduced here can be any type of polynucleotide which is an N-glycoside or C-glycoside of a purine or pyrimidine base. The nucleic acid can be single-, double- or multi-stranded, linear, branched or circular. It can correspond to a molecule occurring in a cell, such as, for example, genomic DNA or messenger RNA (mRNA), or can be produced in vitro, such as complementary DNA (cDNA), antisense RNA (aRNA) or synthetic nucleic acids. The nucleic acid can be made up of few nucleotides or also of several thousand nucleotides.

In the following, the term “reaction vessel with a binding matrix” are to be understood as meaning biochemical separation principles in which a binding matrix which associates with selectively determined substances is arranged in a reaction vessel or a miniaturized column.

In the following, the term “acceleration value” designates the multiple of the acceleration of gravity which is achieved by the speed of rotation of the centrifuge and acts on the goods being centrifuged. This is measured with the parameter g=9.81 ms⁻². 1000× g e.g. designates an acceleration value which is 1000 times the acceleration of gravity. The acceleration value is also called the “centrifugal index” and does not correspond to the speed of rotation of the centrifuge, which as a rule is designated in revolutions per minute (rpm). The acceleration value is determined constructively by the centrifuge drum diameter (effective diameter) and the speed of rotation.

In the following, the term “centrifugation step” is understood as meaning a process step which is distinguished by a definable duration and a definable acceleration value.

This binding matrix preferably comprises an anion exchanger, a silicate substrate, a substrate of plastic or a chitosan-containing substrate.

In the following, the term “silicate substrate” is to be understood as meaning a membrane, a pellet, a packing or a disk of porous silicate which has a large internal surface area and is arranged in the reaction vessel such that a solution introduced into the reaction vessel is driven through the membrane, the pellet, the packing or the disk during application of a vacuum or during centrifugation such that the constituents contained in the solution come into contact with the constituents of the matrix. The silicate substrate is preferably a matrix of silica gel. The silicate substrate can likewise be made of pressed glass fibers or glass beads (“microbeads”). Silicate substrates are used e.g. in the purification kits marketed by the applicant under the trade names QIAprep and RNeasy.

Anion exchangers are adequately known from the prior art. A resin which e.g. interacts with the negatively charged phosphate radicals of the nucleic acid backbone is as a rule used here. The salt concentration and the pH values of the buffers used determine whether the nucleic acid binds to the resin or is eluted from the column.

Such anion exchangers are marketed e.g. by the applicant under the trade names QIAGEN Genomic-tip and Plasmid-tip.

Chitosan has only recently been discussed as a binding agent for biomolecules. This is a copolymer of β-1,4-glycosidically linked N-acetyl-glucosamine radicals and glucosamine radicals. Under physiological conditions, chitosan carries positive net charges and is therefore capable of binding many negatively charged biomolecules, in particular nucleic acids, amino acids, oligo- and polypeptides, fats and fatty acids.

It is moreover particularly preferably envisaged according to the invention that the binding matrix comprises a silicate substrate, and that furthermore the sample containing biomolecules is mixed with at least one chaotropic salt before the centrifugation. The embodiment is suitable in particular for nucleic acids. The separation principle used in this context is based on the “boom principle” process already discussed. In this, a sample containing nucleic acids is introduced into a vessel with a silicate matrix in the presence of a chaotropic salt. The vessel is then centrifuged, or a vacuum is applied. This causes the nucleic acids to bind to the silicate matrix, while all the other constituents of the sample (in particular cell debris, organelles, proteins and the like) pass through the silicate matrix and are discarded. The bound nucleic acids are then eluted with a suitable agent and subjected to further analysis.

Preferably, the following steps are envisaged in this embodiment:
a) arrangement of a column-like reaction vessel with a binding matrix comprising a silicate substrate in a centrifuge, wherein a solution or suspension of a nucleic acid-containing sample and at least one chaotropic salt is prepared in the reaction vessel or introduced into the reaction vessel before or after this step;
b) inclusion of a first centrifugation step at a first acceleration value;
c) inclusion of a second centrifugation step at a second acceleration value which is higher than the first acceleration value;
d) optionally inclusion of further centrifugation steps between step c) and step d) or after step d);
e) optionally inclusion of one or more washing steps; and
f) elution of the nucleic acids bound to the silicate substrate with an elution solution.

In this embodiment, the multi-step centrifugation step is a binding step in which the nucleic acids are bound to the silicate matrix. This embodiment leads to a considerably improved yield of nucleic acids to be purified compared with one-step processes known from the prior art with “spin columns” containing silicate matrices. Alternatively or in addition to this, however, it can also be envisaged that the washing and/or the elution step is designed as several steps in the context of the above protocol.

The washing step or steps are preferably carried out with a wash buffer. This can contain, in particular, ethanol and/or acetone.

The elution solution for elution of the biomolecules, in particular nucleic acids, bound to the binding matrix can be e.g. water (including aqua dist) or a low-molar solution. A weakly concentrated sodium chloride solution e.g. is possible here.

The chaotropic salt is preferably already in solution. Alternatively, the sample containing nucleic acids can be in solution or suspension and a chaotropic salt can then be added. Alternatively in turn, the sample and chaotropic salt can be present as a solid and brought into solution or suspension together.

In the following, the term “column-like reaction vessel” is to be understood as meaning a vessel that is optionally closable at the top and optionally open at the bottom. The reaction vessel contains the silicate matrix described above. A typical example of a reaction vessel in the above sense are the so-called “spin columns” such as are produced and marketed by the applicant. The reaction vessels can preferably be configured such that they can be arranged to fit accurately in a commercially available, somewhat larger reaction vessel, such as e.g. is marketed by Eppendorf. In this case the larger reaction vessel serves as the collection vessel for the liquid passing through the binding matrix.

The processes according to the invention which are mentioned have the common feature that by the combination for the first time of a centrifugation step at a low acceleration value and a centrifugation step at a high acceleration value the yield in the biomolecule purification is increased by up to 20%, as studies by the applicant have shown (see examples). By this means, analytical investigations are facilitated considerably, and in many cases even first made possible; there are cases in which e.g. the amount of nucleic acids in the sample is so low that the yield with conventional purification methods is not sufficient for the nucleic acids to be amplified and/or detected.

The improvements to the yield mentioned are surprising and were not foreseeable by the person skilled in the art. In view of the fact that “column spin” processes to date have always been carried out at a single acceleration value, a two-step centrifugation process when considered superficially seems very unattractive, because this takes a longer time than a one-step centrifugation process.

The process according to the invention can be carried out in a commercially available, manually operable bench centrifuge, such as e.g. is produced by the manufacturer of laboratory equipment Eppendorf and is present in any laboratory working in biosciences. In this case the centrifugation protocol is completed “manually” with at least two centrifugation steps at different acceleration values, i.e. user intervention is necessary for inclusion of the different centrifugation steps.

Needless to say, it is preferably envisaged that the process according to the invention is carried out in an automated and/or programmable centrifuge. It can be envisaged here in particular that the centrifuge already has one or more internally stored centrifugation protocols with at least two centrifugation steps at different acceleration values. Such a centrifuge falls expressly under the scope of protection of the present invention.

The biological sample is particularly preferably a material chosen from the group containing sample material, plasma, body fluids, blood, serum, cells, leukocyte fractions, crust phlogistica, sputum, urine, sperm, feces, forensic samples, smears, puncture samples, biopsies, tissue samples, tissue parts and organs, foodstuff samples, environmental samples, plants and plant parts, bacteria, viruses, viroids, prions, yeasts and fungi, and fragments or constituents of the abovementioned materials, and/or isolated, synthetic or modified proteins, nucleic acids, lipids, carbohydrates, metabolism products and/or metabolites.

In this context, for subsequent analysis of the nucleic acids in or from the biological sample all analysis methods which are known and seem suitable to the person skilled in the art can be employed, preferably methods chosen from the groups including light microscopy, electron microscopy, confocal laser scanning microscopy, laser microdissection, scanning electron microscopy, western blotting, Southern blotting, enzyme-linked immonosorbent assay (ELISA), immunoprecipitation, affinity chromatography, mutation analysis, polyacrylamide gel electrophoresis (PAGE), in particular two-dimensional PAGE, HPLC, polymerase chain reaction (PCR), RFLP analysis (restriction fragment length polymorphism analysis), SAGE analysis (serial analysis of gene expression), FPLC analysis (fast protein liquid chromatography), mass spectrometry, for example MALDI-TOFF mass spectrometry or SELDI mass spectrometry, microarray analysis, LiquiChip analysis, lysis of the activity of enzymes, HLA typing, sequencing, WGA (“whole genome amplification”), RT-PCR, real time PCR or -RT-PCR, RNase protection analysis or primer extension analysis.

Preferably, it is envisaged that the process is preceded by a step for lysis of cells or tissues containing biomolecules.

This lysis step can be e.g. a physical or a chemical lysis. Physical lysis processes which are employed are, in particular, the use of ultrasound, a successive freezing and thawing (“freeze/thaw”), the use of rotating blades, the use of oscillating microbeads, the action of a hypotonic shock, the so-called “French press process” or the so-called “cell bomb process”.

A possible chemical lysis process is, in particular, the use of phenol, chloroform and/or isoamyl alcohol. Enzymatic processes likewise fall under this term, thus e.g. the use of lysozyme for bacteria or the use of β-glucuronidase (“snail gut enzyme”) for yeast.

A special form is alkaline lysis. This is used in particular to isolate plasmid DNA from already lysed bacteria. By addition of NaOH to the cell extract, the hydrogen bridge bonds between the complementary DNA strands of both the chromosomal and the plasmid DNA dissolve, the plasmid DNA being capable of renaturing completely due to its conformation. The chromosomal DNA, which has been broken into pieces by the individual preparation steps, cannot renature after neutralization of the pH with potassium acetate and glacial acetic acid, and DNA double strands with only short complementary regions form and due to the non-aligned joining of many DNA single strands a tangled mass of DNA forms. This can be centrifuged off relatively easily together with the NaOH which has precipitated out due to the neutralization. In this centrifugation step, cell membrane and cell wall constituents as well as proteins are furthermore deposited as a pellet. The plasmid DNA is in the supernatant after the centrifugation.

It is furthermore particularly preferably envisaged that the chaotropic salt used according to the invention is a salt or a mixture of salts chosen from the group containing guanidinium hydrochloride, guanidinium thiocyanate, guanidinium iodide, urea, ammonium sulfate, sodium iodide, potassium iodide, sodium perchlorate, sodium (iso)thiocyanate and guanidium thiocyanate.

Chaotropic salts are salts which have a high affinity for water and therefore form a hydration shell. In the presence of these salts, the hydrophobic interactions in proteins are destabilized because the solubility of the hydrophobic side chains increases, and the protein denatures. Nucleic acids, such as DNA and RNA, on the other hand, are not impaired because no hydrophobic interactions are necessary for stabilization thereof In addition, the cations of chaotropic salts in high concentrations satisfy the negative charges on the surface of silicates, in particular in silicate matrices, and generate a positive net charge, which considerably forces the binding of the nucleic acids to the silicate matrices.

The first centrifugation step of the process is preferably carried out at an acceleration value in the range of between 5-2000×g. Particularly suitable acceleration values are 10×g, 27×g, 50×g, 150×g, 300×g, 500×g, 800×g, 1000×g and 1500×g. This centrifugation step can have, for example, a duration of 5 s-20 min. A duration of 10 s-10 min is particularly preferred. A duration of 30 s-5 min is particularly preferred.

The second centrifugation step of the process is preferably carried out at an acceleration value in the range of between 100-25000×g. Particularly suitable acceleration values are 180×g, 610×g, 1000×g, 2500×g, 8000×g, 12000×g and/or 17000×g. This centrifugation step can likewise have, for example, a duration of 5s -20 min. A duration of 10 s-10 min is particularly preferred. A duration of 30 s-5 min is very particularly preferred.

As can be seen from the above description, the value ranges for the acceleration values of the first and the second centrifugation step overlap. However, it must be ensured according to the invention that the acceleration value of the first centrifugation step is always below the acceleration value of the second centrifugation step.

It is furthermore preferably envisaged that the reaction vessels are centrifuged in a centrifuge rotor of the “swing-out type”. In such rotors, the centrifugation angle required is only established when the rotor is set in motion. The process according to the invention indeed also has the said improvements in yield when fixed angle rotors are used, but centrifuge rotors of the “swing-out type” are preferably employed if substances are to be introduced into reaction or centrifugation vessels already arranged in the rotor, e.g. by pipetting or with the aid of a pipetting robot.

Particularly preferably, it is envisaged that the individual steps of the process proceed by an automated procedure. For this, the applicant has developed inter alia his own device which combines the functions of a pipetting robot and a programmable centrifuge. With the aid of such an automated process, the laboratory throughput can be increased considerably and at the same time assignment errors can be largely avoided. Both factors play an important role precisely in clinical, forensic, epidemiological and population genetics investigations.

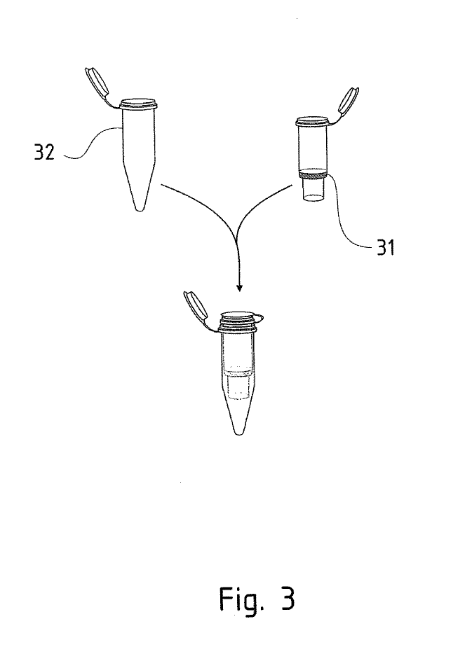

A reaction vessel containing a binding matrix for use in a process for the purification of biomolecules, preferably nucleic acids, from a sample is furthermore provided. Such a reaction vessel is shown e.g. in FIG. 3.

A composition for use in a process for the purification of biomolecules, preferably nucleic acids, from a sample is furthermore provided according to the invention, the composition comprising at least one constituent chosen from the group containing alkaline agents, phenol, lytic enzymes, isoamyl alcohol, chloroform, Chaotropic salts, alcohols, water and inorganic or organic salts.

This composition can be e.g. a lysis buffer (phenol, lytic enzymes, isoamyl alcohol, chloroform), a binding buffer (Chaotropic salts), a wash buffer (alcohols, inorganic or organic salts) or an elution buffer (inorganic or organic salts).

A kit of parts comprising at least one such composition is furthermore provided according to the invention. Particularly preferably, this kit comprises at least a reaction vessel as mentioned above and furthermore reagents for analysis of biomolecules in or from a biological sample or for analysis of the morphology of a biological sample.

Reagents for analysis of biomolecules which can be employed here are, in particular, reagents for detection and quantification of nucleic acids, amino acids, oligopeptides, polypeptides, monosaccharides, oligosaccharides, polysaccharides, fats, fatty acids and/or lipids. The person skilled in the art can discover such reagents from the technical literature without his own inventive step. Such reagents are often already obtainable ready-made as kits for the particular biomolecules to be analyzed. These reagents include, in particular, dyestuffs for staining cells or cell constituents, antibodies, optionally labeled with fluorescent dyestuffs or enzymes, an absorption matrix, such as, for example, DEAE cellulose or a silica membrane, substrates for enzymes, agarose gels, polyacrylamide gels, solvents, such as ethanol or phenol, aqueous buffer solutions, RNase-free water, lysis reagents, alcoholic solutions and the like.

In this context, the composition can already be introduced into the vessel.

However, it is also conceivable that the kit includes a metering device as a further constituent, which is filled with the composition and by means of which defined portions of the composition can be introduced into the vessel, preferably under sterile conditions. Such a metering device can be constructed, for example, in the form of a soap dispenser.

A device for purification of biomolecules, preferably nucleic acids, from a sample, comprising a centrifuge, is moreover provided according to the invention, which is characterized in that the device comprises means which make it possible for at least two centrifugation steps with acceleration values at different levels to be included by an automated procedure during a centrifugation without user intervention. For this purpose, a microprocessor control which has a storage device in which multi-step centrifugation protocols are stored and/or can be stored is as a rule necessary.

A centrifugation device which accordingly comprises means for carrying out the process described above for purification of biomolecules from a sample is likewise provided according to the invention. In this context, a microprocessor control which makes it possible for at least two centrifugation steps with acceleration values of different levels to be included by an automated procedure during a centrifugation without user intervention is intended in particular.

Such a centrifugation device comprises means for carrying out the process according to the invention by in an automated procedure. This includes inter alia, in addition to the microprocessor control mentioned, e.g. a pipetting robot.

A purified nucleic acid which can be prepared with a process, a composition, a kit and/or a device according to the present invention is furthermore provided according to the invention. This nucleic acid is, in particular, plasmid, genomic, viral and mitochondrial DNA or mRNA, siRNA, miRNA, rRNA, snRNA, t-RNA and hnRNA.

### FIGURES AND EXAMPLES

The present invention is explained in more detail by the examples and figures shown and discussed in the following. It is to be noted here that the examples have only a descriptive character and are not intended to limit the invention in any form.

### Example 1

### Basic Procedure (One-Step Process According to the Prior Art)

Bacteria colonies grown on an agar plate and containing a plasmid to be isolated are picked, suspended in 3 ml each of LB liquid culture medium and incubated at 37° C. overnight for multiplication of the. The saturated 3 ml bacteria overnight cultures are pelleted in a bench centrifuge at 13000 rpm. The plasmid DNA is isolated by a modified standard protocol from Qiagen by the method of Birnboim. The supernatant of the bacteria culture is removed and discarded. 250 μl of buffer P1 (Qiagen) are added to the pellet and the pellet is resuspended. The bacteria are lysed by addition of 250 μl of buffer P2 (Qiagen) and shaking carefully 4-5 times (alkaline lysis); the lysis reaction should not last longer than 5 min, because otherwise the genomic DNA is mobilized. The lysis reaction is therefore stopped by addition of 350 μl of buffer N3 (Qiagen) and immediate gentle shaking. The lysed bacteria wall constituents are pelleted at 13000 rpm for 10 min.

The plasmids in the supernatant are carefully removed and pipetted into a prepared Qiagen spin column. The subsequent procedure is then as follows:

### Example 2A

### Comparison of the DNA Yield Between the One-Step and Two-Step Centrifugation Process (Binding Step)

3 ml of a bacteria culture (DH10B) which contains the plasmid puc 19 were harvested and lysed as described above and transferred into spin columns (QIAprep model), and then subjected to a conventional one-step (“manual 1-step protocol”) or two-step (“manual 2-step binding”) centrifugation process. The process parameters were as follows:

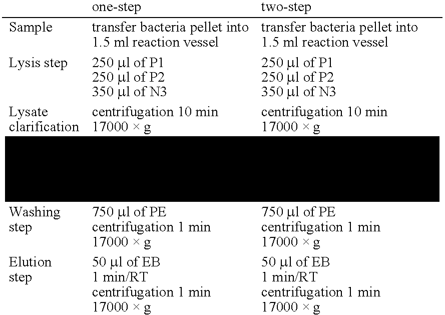

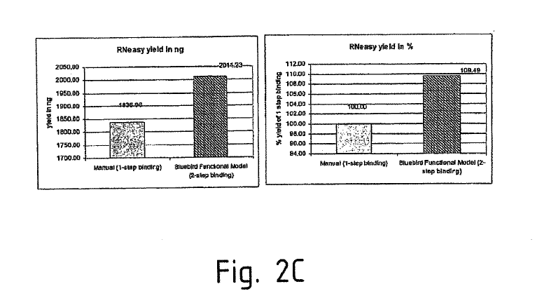

The essential differences in the centrifugation protocol have a gray background. The buffers P1, P2, N2, PE and EB are constituents of the QIAprep Kit. The yield of plasmid DNA was then investigated. In each case 8 parallel experiments were carried out, and the results were evaluated statistically and are shown in FIG. 2A. While a DNA yield of 8454 ng was achieved with the one-step process, a yield of 9540 ng was achieved with the two-step process. The differences are significant. It can be clearly seen that the DNA yield with the two-step process was higher by approx. 13%.

### Example 2B

### Comparison of the DNA Yield Between the One-Step and Two-Step Centrifugation Process (Washing Step)

Similar differences were to be found when instead of the binding step the washing step was designed as two stages, for example as shown in the following table:

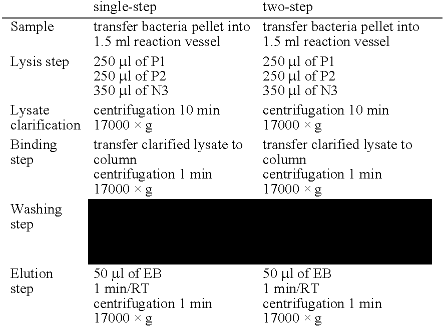

The essential differences in the centrifugation protocol have a gray background. In each case 8 parallel experiments were carried out, and the evaluation was performed as in the above example. The results are shown in FIG. 2B. While a DNA yield of 4022 ng was achieved with the one-step process, a yield of 4803 ng was achieved with the two-step process. The differences are significant. It can be clearly seen that the DNA yield with the two-step process was higher by approx. 19%.

### Example 2C

### Comparison of the RNA Yield Between the One-Step and Two-Step Centrifugation Process

Jurkat cells were lysed with a standard process (Qiagen RNeasy) and transferred into spin columns (RNeasy model), and then subjected to a conventional one-step (“manual standard protocol”) or two-step (“manual 2-step binding”) centrifugation process. The process parameters were as follows:

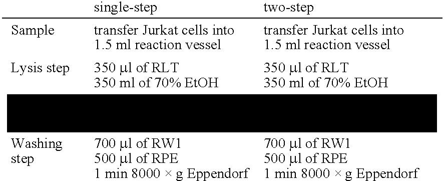

The differences in the centrifugation protocol have a gray background. The buffers RPE, RW1 and RLT are constituents of the RNeasy Kit. The yield of RNA was then investigated. In each case 8 parallel experiments were carried out, and the results were evaluated statistically and are shown in FIG. 2C.

While an RNA yield of 1836 ng was achieved with the one-step process, a yield of 2011 ng was achieved with the two-step process. The differences are significant. It can be clearly seen that the RNA yield with the two-step process was higher by approx. 9%.

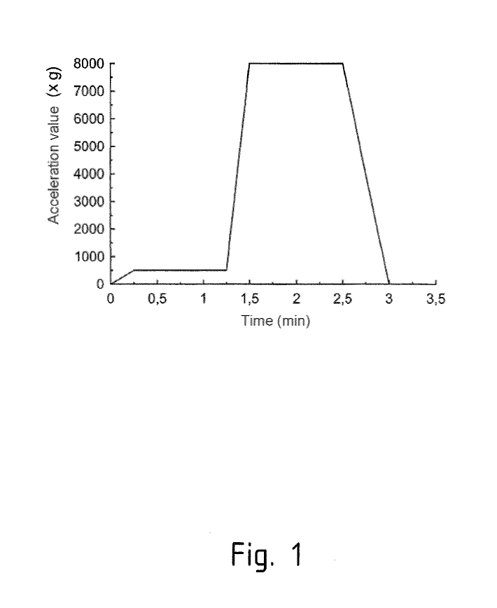

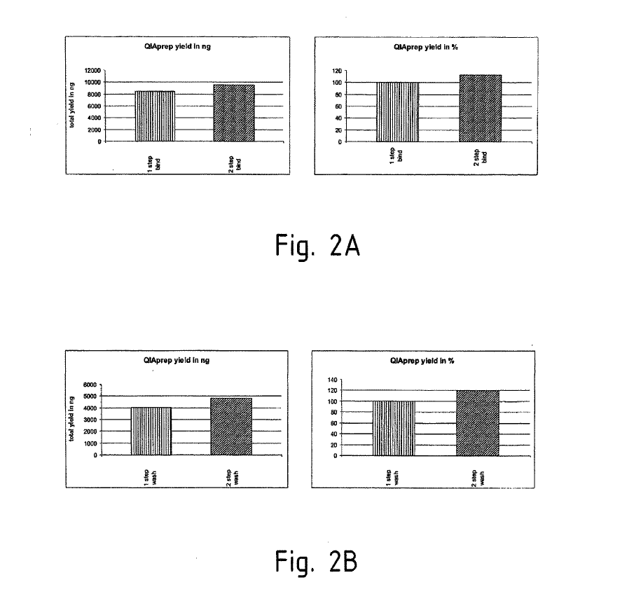

FIG. 1 shows as a time graph the course, by way of example, of a centrifugation protocol according to the process according to the invention with a multi-stage centrifugation step. In the example shown, the multi-stage centrifugation step is a binding step in which the biomolecules are bound to the binding matrix by centrifugation.

For this, the binding buffer is added to the sample to be purified and centrifugation is then initially carried out at 500×g for 1 min. The centrifuge then accelerates until an acceleration value of 8000×g is reached, and the sample is centrifuged at this value for a further 75 sec. During this procedure the nucleic acids bind to the silicate matrix, while all the remaining constituents pass through the silicate matrix and can be discarded. Washing is then carried out with a wash buffer, and the nucleic acids are washed from the column with an elution buffer and collected.

FIG. 2 shows the results of the experiments described in Example 2A, 2B and 2C. In this, on the one hand the absolute yields of nucleic acid in ng are shown, and on the other hand the performance advantage of the particular two-step process in % is shown.

FIG. 3 shows a reaction vessel 30, containing a silicate matrix 31, for use in a process according to the invention. After the reaction vessel 30 has been charged with a solution or suspension of a nucleic acids-containing sample and at least one chaotropic salt or such a solution or suspension has been prepared in the reaction vessel, the reaction vessel is positioned in an accurately fitting larger collection vessel 32. The combination of the two vessels is now subjected in a centrifuge, not shown, to the centrifugation protocol according to the invention with a first centrifugation step at a first acceleration value and second centrifugation step at a second acceleration value which is higher than the first acceleration value. During this procedure, the nucleic acids bind to the silicate matrix, while all the remaining constituents pass through the silicate matrix and can be discarded. Washing is then carried out with a wash buffer, and the nucleic acids are washed from the column with an elution buffer and collected.

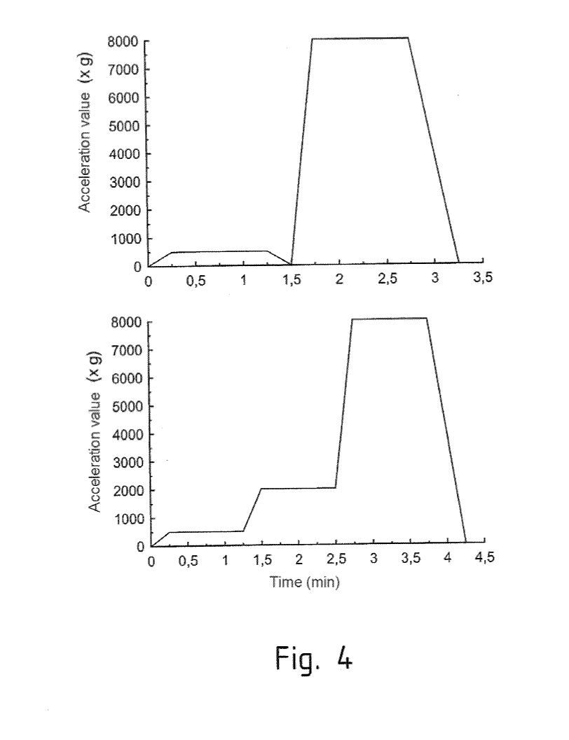

FIG. 4 shows as a time graph, like FIG. 1, the course, by way of example, of two further centrifugation protocols according to the process according to the invention. In the protocol shown at the top, the centrifuge is stopped briefly between the individual centrifugation steps at various acceleration values. The descriptions given for FIG. 1 otherwise apply.

In the protocol shown at the bottom, a further centrifugation step at an intermediate acceleration value is included between the first and the second m centrifugation step. It is conceivable that still further centrifugation steps are included, which would give the time graph a more or less staircase-like appearance.

1. A process for the purification of biomolecules from a sample, comprising the following steps:
a) arrangement of a reaction vessel with a binding matrix in a centrifuge, wherein a solution or suspension of a sample comprising biomolecules is prepared in the reaction vessel and/or introduced into the reaction vessel before or after this step; and
b) inclusion of at least one multi-stage centrifugation step comprising at least a first centrifugation step at a first acceleration value and at least a second centrifugation step at a second acceleration value which is higher than the first acceleration value; wherein
c) step b) can be a binding step, a washing step and/or an elution step.

2. The process as claimed in claim 1, wherein the biomolecules are at least one selected from the group consisting of nucleic acids, amino acids, oligopeptides, polypeptides, monosaccharides, oligosaccharides, polysaccharides, fats, fatty acids and lipids.

3. The process as claimed in claim 1, wherein the binding matrix comprises an anion exchanger, a silicate substrate, a substrate of plastic and/or a chitosan-containing substrate.

4. The process as claimed in claim 3, wherein the binding matrix comprises a silicate substrate, and the sample containing biomolecules is mixed with at least one chaotropic salt before the centrifugation.

5. The process as claimed in claim 1, wherein the process is preceded by a step for lysis of cells and/or tissues comprising biomolecules.

6. The process as claimed in claim 4, wherein the chaotropic salt is a salt or a mixture of salts chosen from guanidinium hydrochloride, guanidinium thiocyanate, guanidinium iodide, urea, ammonium sulfate, sodium iodide, potassium iodide, sodium perchlorate, sodium (iso)thiocyanate and/or guanidium thiocyanate.

7. The process as claimed in claim 1, wherein the first centrifugation step is carried out at an acceleration value in the range of from 5-2000×g.

8. The process as claimed in claim 1, wherein the second centrifugation step is carried out at an acceleration value in the range of from 100-25000×g.

9. The process as claimed in claim 1, wherein the reaction vessels are centrifuged in a centrifuge rotor of the “swing-out type”.

10. The process as claimed in claim 1, the wherein individual steps of the process proceed by an automated procedure.

11. A reaction vessel containing a binding matrix suitable for use in a process for the purification of biomolecules from a sample as claimed in claim 1.

12. A composition suitable for use in a process for the purification of biomolecules from a sample as claimed in claim 1, wherein the composition comprises at least one constituent chosen from alkaline agents, phenol, lytic enzymes, isoamyl alcohol, chloroform (lysis buffer), chaotropic salts (binding buffer), alcohols (binding buffer) and/or inorganic or organic salts (elution buffer).

13. A kit of parts comprising at least one composition as claimed in claim 12.

14. The kit of parts as claimed in claim 13, comprising
(a) at least a reaction vessel as claimed in claim 11, and
(b) reagents for analysis of biomolecules in or from a biological sample and/or for analysis of the morphology of a biological sample.

15. A device suitable for purification of biomolecules from a sample, comprising a centrifuge, wherein the device comprises means which make it possible for at least two centrifugation steps with acceleration values at different levels to be included by an automated procedure during a centrifugation without user intervention.

16. A centrifugation device comprising means for carrying out a process for the purification of biomolecules from a sample as claimed in claim 1.

17. The centrifugation device as claimed in claim 16, comprising means for automatically carrying out said process.

18. A purified nucleic acid which can be prepared by a process as claimed in claim 1.
